(19) United States
(12) Patent Application Publication
(10) Pub. No.: US 2022/0282189 A1
(43) Pub. Date: Sep. 8, 2022

(54) BLEACH ACTIVATOR WITH A CATIONIC GROUP AND WASHING OR CLEANING AGENT CONTAINING SAID BLEACH ACTIVATOR

(71) Applicant: Henkel AG & Co. KGaA, Duesseldorf (DE)

(72) Inventors: Sascha Schaefer, Duesseldorf (DE); Svenja Neuhaus, Duesseldorf (DE); Antje Gebert-Schwarzwaelder, Duesseldorf (DE); Christa Junkes, Duesseldorf (DE); Arne Jansen, Duesseldorf (DE)

(73) Assignee: Henkel AG & Co. KGaA, Duesseldorf (DE)

(21) Appl. No.: 17/749,459

(22) Filed: May 20, 2022

Related U.S. Application Data
(63) Continuation of application No. PCT/EP2020/082195, filed on Nov. 16, 2020.

(30) Foreign Application Priority Data
Nov. 20, 2019 (DE) 102019217849.9

(51) Int. Cl. C11D 3/39; C11D 3/395; C11D 3/28; C11D 3/34; C11D 11/00; C07D 403/10; C07D 417/10

(52) U.S. Cl. CPC C11D 3/3927; C11D 3/3951; C11D 3/28; C11D 3/349; C11D 3/3953; C11D 3/3942; C11D 3/3947; C11D 3/3956; C11D 11/0017; C11D 11/0023; C07D 403/10; C07D 417/10

(57) ABSTRACT

The oxidizing and bleaching effect of peroxygen compounds at low temperatures was to be improved. This was substantially achieved through the use of organic peroxoacids under perhydrolysis conditions with compounds which form cationic groups and in which a quaternary nitrogen atom is part of a heterocyclic 5-membered functional group.

### FIELD OF INVENTION

The present invention relates to compounds forming specific cationic organic peracids under perhydrolysis conditions, to the use of said compounds for activating peroxygen compounds in connection with the bleaching of stains when washing textiles and cleaning hard surfaces, and to washing and cleaning agents containing said compounds.

### BACKGROUND

Inorganic peroxygen compounds, in particular hydrogen peroxide and solid peroxygen compounds which dissolve in water to release hydrogen peroxide, such as sodium perborate and sodium carbonate perhydrate, have long been used as oxidizing agents for disinfecting and bleaching purposes. The oxidizing effect of these substances depends largely on the temperature in dilute solutions; for example, with H₂O₂ or alkali perborate in alkaline bleaching liquors, sufficiently rapid bleaching of soiled textiles is achieved only at temperatures above approximately 80° C. At lower temperatures, the oxidizing effect of the inorganic peroxygen compounds can be improved by adding what are known as bleach activators, which are able to provide peroxocarboxylic acids under the perhydrolysis conditions mentioned and which have become known in the literature for which numerous proposals, especially from the classes of N- or O-acyl compounds, for example polyacylated alkylenediamines, in particular tetraacetylethylenediamine, acylated glycolurils, in particular tetraacetylglycoluril, N-acylated hydantoins, hydrazides, triazoles, hydrotriazines, urazoles, diketopiperazines, sulphurylamides and cyanurates, as well as carboxylic acid anhydrides, in particular phthalic acid anhydride and alkyl succinic acid anhydride, carboxylic acid esters, in particular sodium nonanoyloxybenzenesulfonate, sodium isononanoyloxybenzenesulfonate, O-acylated sugar derivatives, such as pentaacetylglucose, and N-acylated lactams, such as N-benzoylcaprolactam. By adding these substances, the bleaching effect of aqueous peroxide liquors can be increased to such an extent that substantially the same effects occur at temperatures around 60° C. as with the peroxide liquor alone at 95° C.

In the pursuit of energy-saving washing and bleaching processes, application temperatures well below 60° C., in particular below 45° C. down to cold water temperature, have gained importance in recent years.

At low temperatures, the effect of the previously known activator compounds usually decreases noticeably.

The international patent application WO 96/06915 A1 discloses bleach activators which, under perhydrolysis conditions, form organic peroxoacids with a cationic group.

### SUMMARY OF THE INVENTION

Surprisingly, it has now been found that under perhydrolysis conditions organic peroxo acids with compounds forming cationic groups, in which a quaternary N atom is part of a heterocyclic 5-membered functional group, are particularly suitable for increasing the bleaching effect of peroxygen-containing washing and cleaning agents.

The invention therefore relates to a bleach-activating compound of general formula (I),

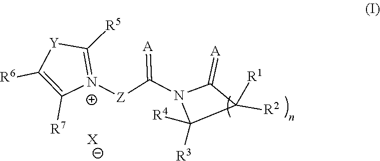

in which

A represents O, S, Se;

Z represents an optionally substituted C₁₋₃₀ alkylene, C₃₋₃₀ cycloalkylene, arylene, alkylenearylene or arylenealkylene functional group;

X⁻ represents an anion, in particular chloride, bromide, iodide, tosylate, mesylate, triflate, sulfate, carbonate and/or phosphate;

Y represents NR⁸, O, S, Se;

R¹, R², R³, R⁴, R⁵, R⁶, R⁷ and R⁸ represent, independently of one another, H, a C₁₋₃₀ alkyl, C₃₋₃₀ cycloalkyl, aryl, alkylaryl or arylalkyl functional group; and

n represents a number in the range from 0 to 12, in particular from 1 to 12.

In the specified hydrocarbon functional groups, 1 or more non-adjacent carbon atoms not bonded to a heteroatom can be replaced by a heteroatom, in particular N, O, S and/or Se. If X⁻ is a polyvalent anion, correspondingly several of the units which contain the cationic heterocycle and which form the remaining part of the compound according to formula (I) are present per anion X. R¹, R², R³, R⁴, R⁵, R⁶, R⁷ and/or R⁸ are preferably H, and/or A is preferably O, and/or n is preferably a number in the range from 2 to 4. As shown in the following formula (II), Y is preferably NR⁸ and Z is preferably derived from an optionally substituted alkylbenzene unit in which a C atom of the aromatic ring is bonded to the unit C=A, a C atom of the alkyl functional group is bonded to the shown N atom of the five-membered heterocycle and the methyl group is the preferred alkyl functional group:

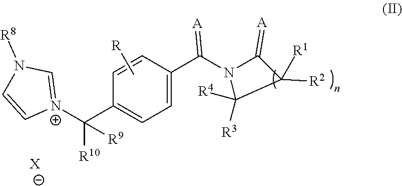

For the compounds of formula (II), the definitions given for formula (I) for A, n, R¹ to R⁸ and X⁻ also apply; R, R⁹ and R¹⁰ represent, independently of one another, H, a C₁₋₃₀ alkyl, C₃₋₃₀ cycloalkyl, aryl, alkylaryl or arylalkyl functional group, where 1 or more non-adjacent carbon atoms not bonded to a heteroatom can also be replaced by a heteroatom, in particular N, O, S and/or Se, in these functional groups. Preferably R, R⁹ and/or R¹⁰ are H.

The invention also relates to the use of compounds of general formula (I) for increasing the cleaning performance of peroxygen-containing and in particular surfactant-containing washing or cleaning agents in aqueous liquor.

The use according to the invention substantially consists in creating conditions under which the peroxygen compound and the compound of general formula (I) can react with one another, with the aim of obtaining secondary products with a greater oxidizing effect. Such conditions exist in particular when the reaction partners meet in an aqueous system. This can be achieved by separately adding the peroxygen compound and the compound of general formula (I) to a liquor which may optionally contain washing or cleaning agents. However, it is particularly advantageously carried out using a washing or cleaning agent which contains the compound of formula (I) and a peroxidic oxidizing agent. The peroxygen compound can also be added separately, in bulk or as a preferably aqueous solution or suspension, to the aqueous system if a washing or cleaning agent free of peroxygen compounds is used. Depending on the intended use, the conditions can be varied widely. In addition to purely aqueous solutions, mixtures of water and suitable organic solvents can also be used as the reaction medium.

The invention also relates to a method for washing laundry and to a method for cleaning hard surfaces, comprising the method steps of (a) providing an aqueous liquor containing H₂O₂ or an in particular inorganic peroxygen compound according to general formula (I) which provides H₂O₂ in water, and (b) bringing this liquor into contact with textiles to be washed or hard surfaces to be cleaned. Preferably, the contact between the aqueous liquor and the object to be washed or cleaned is made at temperatures in the range of from 20° C. to 40° C., in particular from 20° C. to 30° C. The object to be washed or cleaned preferably remains in contact with the aqueous liquor over a period of from 20 minutes to 120 minutes, in particular from 30 minutes to 90 minutes. The methods according to the invention can be carried out manually or by means of conventional devices, for example washing machines or dishwashers. In the context of a use according to the invention and a method according to the invention, the amounts of peroxygen compounds used are generally selected such that between 10 ppm and 10% active oxygen, preferably between 50 ppm and 5,000 ppm active oxygen, are present in the liquor.

A peroxygen compound, together with a compound according to general formula (I), is preferably used for bleaching stains, in particular tea, when washing textiles, in particular in an aqueous, surfactant-containing liquor. The expression “bleaching of stains” is to be understood in its broadest sense and includes the bleaching of colored dirt on the textile, the bleaching of colored dirt in the washing liquor that has been detached from the textile, and the oxidative destruction of textile dyes in the washing liquor that detach from textiles under the washing conditions before they can be absorbed onto textiles of different colors.

Another preferred application form according to the invention is the use of a peroxygen compound together with the compound according to general formula (I) in in particular aqueous, surfactant-containing cleaning solutions for hard surfaces, in particular for dishes, for bleaching colored stains, for example tea. Here, too, the term bleaching is understood to mean both the bleaching of dirt on the hard surface, in particular tea, and the bleaching of dirt that has been detached from the hard surface in the cleaning liquor, for example a dishwashing liquor.

The uses according to the invention for increasing the cleaning performance of washing and cleaning agents and for bleaching colored stains can be implemented particularly easily by introducing the compound of general formula (I) into an in particular surfactant-containing aqueous liquor which also contains peroxygen compound and the textile to be cleaned or the object to be cleaned with a hard surface, the sequence of introduction of the compound according to general formula (I), peroxygen compound and the textile or object with a hard surface being arbitrary, or by applying an in particular surfactant-containing aqueous liquor which contains the compound of general formula (I) and peroxygen compound onto the textile to be cleaned or the hard surface to be cleaned. The compound of general formula (I) is preferably used as a constituent of a washing or cleaning agent which particularly preferably also contains the peroxygen compound. The invention therefore also relates to washing or cleaning agents containing a compound according to general formula (I). A washing or cleaning agent preferably contains 0.01 wt. % to 50 wt. %, in particular 0.1 wt. % to 25 wt. % and particularly preferably 1 wt. % to 10 wt. %, of compound according to general formula (I). The percentages by weight relate to the total washing or cleaning agent; this also applies to all of the following percentages by weight, unless expressly stated otherwise. In aqueous washing or cleaning liquors, quantities of 1 g/l to 10 g/l, in particular 2 g/l to 5 g/l, of such agents are preferably used. The pH of the washing and cleaning liquors is generally not critical and can, for example, be in the range of from pH 4 to pH 14; it is preferably in the range of from pH 6 to pH 12, in particular from pH 7 to pH 10.

The preferred embodiments of the compounds according to formula (I) mentioned above are also preferred for use in the methods, uses and agents according to the invention.

The compounds of formula (I) and the peracids formed therefrom under perhydrolysis conditions are generally more water-soluble than analogous compounds in which the cationic substituent is absent or which instead have an anionic substituent, and they have a higher affinity to the surface to be cleaned, in particular if it is made of cotton or contains cotton.

The washing and cleaning agents according to the invention, which may be present in particular as powdery solids, in compacted particle form, as homogeneous solutions or as suspensions or dispersions, can in principle contain all known ingredients common in such agents, in addition to the bleach-enhancing compound and preferably peroxygen compound used according to the invention. The washing and cleaning agents according to the invention can in particular contain builders, surface-active surfactants, water-miscible organic solvents, enzymes, sequestering agents, electrolytes, pH regulators and other auxiliaries, such as optical brighteners, graying inhibitors, dye transfer inhibitors, foam regulators, additional peroxygen activators and dyes and fragrances.

Suitable organic or inorganic peroxygen compounds include organic peracids or peracid salts of organic acids, such as phthalimidopercaproic acid, perbenzoic acid or salts of diperdodecanedioic acid, but in particular inorganic peroxygen compounds such as hydrogen peroxide and, under the cleaning conditions, inorganic salts that release hydrogen peroxide, such as perborate, percarbonate and/or persilicate, and hydrogen peroxide inclusion compounds such as H₂O₂ urea adducts. Hydrogen peroxide can also be produced by means of an enzymatic system, i.e. an oxidase and the substrate thereof. If solid peroxygen compounds are intended to be used, these may be used in the form of powders or granules, which may also be coated in a manner known in principle. The peroxygen compounds can be added to the washing or cleaning liquor as such or in the form of the agents containing them, which in principle can contain all conventional washing or cleaning agent constituents. Particularly preferably, alkali percarbonate, alkali perborate monohydrate or hydrogen peroxide in the form of aqueous solutions containing from 3 wt. % to 10 wt. % hydrogen peroxide are used. Here and in the following, sodium is the preferred alkali metal. If a washing or cleaning agent according to the invention contains peroxygen compounds, in particular sodium percarbonate, these are preferably present in amounts of from 1 wt. % to 15 wt. %, in particular from 4 wt. % to 6 wt. %.

In particular, compounds which produce, under perhydrolysis conditions, optionally substituted perbenzoic acid and/or aliphatic peroxycarboxylic acids having 1 to 12 C atoms, in particular 2 to 4 C atoms, alone or in mixtures, are used as a further compound that produces peroxycarboxylic acid under perhydrolysis conditions. The bleach activators mentioned at the outset, which have O- and/or N-acyl groups in particular of the stated number of C atoms and/or optionally substituted benzoyl groups, are suitable. Preferred are polyacylated alkylenediamines, in particular tetraacetylethylenediamine (TAED), acylated glycolurils, in particular tetraacetylglycoluril (TAGU), acylated triazine derivatives, in particular 1,5-diacetyl-2,4-dioxohexahydro-1,3,5-triazine (DADHT), N-acylimides, in particular N-nonanoylsuccinimide (NOSI), acylated phenol sulfonates or carboxylates or the sulfonic or carboxylic acids thereof, in particular nonanoyl or isononanoyl or lauroyl oxybenzene sulfonate (NOB S or iso-NOBS or LOBS), 4-(2-decanoyloxyethoxycarbonyloxy)-benzene sulfonate (DECOBS) or decanoyloxybenzoate (DOBA), acylated polyhydric alcohols, in particular triacetin, ethylene glycol diacetate and 2,5-diacetoxy-2,5-dihydrodrofuran and acetylated sorbitol and mannitol and mixtures thereof (SORMAN), acylated sugar derivatives, in particular pentaacetylglucose (PAG), pentaacetylfructose, tetraacetylxylose and octaacetyl lactose, acetylated, optionally N-alkylated glucamine and gluconolactone, and/or N-acylated lactams, for example N-benzoyl-caprolactam. A washing or cleaning agent according to the invention is preferably free from further compounds which produce peroxycarboxylic acid under perhydrolysis conditions.

In addition to, or instead of, the further compounds mentioned which form peroxycarboxylic acids under perhydrolysis conditions, other bleach activating compounds, such as nitriles, from which perimidic acids may form, may be present. These include in particular aminoacetonitrile derivatives having a quaternized nitrogen atom according to the formula

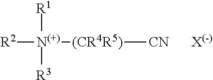

in which R¹ represents —H, —CH₃, a C₂₋₂₄ alkyl or alkenyl functional group, a substituted C₁₋₂₄ alkyl or C₂₋₂₄ alkenyl functional group having at least one substituent from the group —Cl, —Br, —OH, —NH₂, —CN and —N⁽⁺⁾—CH₂—CN, an alkyl or alkenylaryl functional group having a C₁₋₂₄ alkyl group, or a substituted alkyl or alkenylaryl functional group having at least one, preferably two, optionally substituted C₁₋₂₄ alkyl group(s) and optionally further substituents on the aromatic ring, R² and R³ are selected, independently of one another, from —CH₂—CN, —CH₃, —CH₂—CH₃, CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH, where n=1, 2, 3, 4, 5 or 6, R⁴ and R⁵ have, independently of one another, the meaning specified above for R¹, R² or R³, where at least 2 of the functional groups mentioned, in particular R² and R³, also including the nitrogen atom and optionally other heteroatoms, can be linked to one another in a ring-closing manner and then preferably form a morpholino ring, and X is a charge-balancing anion, preferably selected from benzene sulfonate, toluene sulfonate, cumene sulfonate, the C₉₋₁₅ alkylbenzene sulfonates, the C₁₋₂₀ alkyl sulfates, the C₈₋₂₂ carboxylic acid methyl ester sulfonates, sulfate, hydrogen sulfate, and mixtures thereof, may be used.

The presence of bleach-catalyzing transition metal complexes is also possible. These are preferably selected from the cobalt, iron, copper, titanium, vanadium, manganese and ruthenium complexes. Suitable ligands in the transition metal complexes are both inorganic and organic compounds, which include, in addition to carboxylates, in particular compounds having primary, secondary and/or tertiary amine and/or alcohol functions, such as pyridine, pyridazine, pyrimidine, pyrazine, imidazole, pyrazole, triazole, 2,2′-bispyridylamine, tris-(2-pyridylmethyl)amine, 1,4,7-triazacyclononane, 1,4,7-trimethyl-1,4,7-triazacyclononane, 1,5,9-trimethyl-1,5,9-triazacyclododecane, (bis-((1-methylimidazol-2-yl)-methyl))-(2-pyridylmethyl)amine, N,N′-(bis-(1-methylimidazol-2-yl)-methyl)ethylenediamine, N-bis-(2-benzimidazolylmethyl)aminoethanol, 2,6-bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-bis-(bis-(2-pyridylmethyl)aminomethyl)-4-methylphenol, 1,3-bis-(bis-(2-benzimidazolylmethyl)aminomethyl)benzene, sorbitol, mannitol, erythritol, adonitol, inositol, lactose, and optionally substituted salens, porphins and porphyrins. The inorganic neutral ligands include in particular ammonia and water. If not all coordination sites of the transition metal central atom are occupied by neutral ligands, the complex contains further, preferably anionic, ligands, of these in particular mono- or bidentate ligands. These include in particular the halides such as fluoride, chloride, bromide and iodide, and the (NO₂)⁻ group, i.e. a nitro ligand or a nitrito ligand. The (NO₂)⁻ group may also be chelated to a transition metal or it may asymmetrically bridge or η₁-O-bridge two transition metal atoms. In addition to the ligands mentioned, the transition metal complexes to be used according to the invention in the activator system may carry further, generally more simple ligands, in particular mono- or polyvalent anion ligands. For example, nitrate, acetate, trifluoroacetate, formate, carbonate, citrate, perchlorate, and complex anions such as hexafluorophosphate are suitable. The anion ligands are intended to ensure charge balance between the transition metal central atom and the ligand system. The presence of oxo ligands, peroxo ligands and imino ligands is also possible. In particular, such ligands can also have a bridging effect, such that polynuclear complexes are produced. In the case of bridged, binuclear complexes, the two metal atoms in the complex do not need to be the same. The use of binuclear complexes in which the two transition metal central atoms have different oxidation numbers is also possible. If anion ligands are missing or the presence of anionic ligands does not result in charge balance in the complex, anionic counterions which neutralize the cationic transition metal complex are present in the transition metal complex compounds. These anionic counterions include in particular nitrate, hydroxide, hexafluorophosphate, sulfate, chlorate, perchlorate, the halides such as chloride or the anions of carboxylic acids such as formate, acetate, benzoate, citrate or oxalate. Examples of such additional transition metal complex compounds are Mn(IV)₂(μ-O)₃(1,4,7-trimethyl-1,4,7-triazacyclononane)-di-hexafluorophosphate, [N,N′-bis[(2-hydroxy-5-vinylphenyl)-methylene]-1,2-diaminocyclohexane]-manganese-(III)-chloride, [N,N′-bis[(2-hydroxy-5-nitrophenyl)methylene]-1,2-diaminocyclohexane]-manganese-(III)-acetate, [N,N′-bis[(2-hydroxyphenyl)methylene]-1,2-phenylenediamine]-manganese-(III)-acetate, [N,N′-bis[(2-hydroxyphenyl)methylene]-1,2-diaminocyclohexane]-manganese-(III)-chloride, [N,N′-bis[(2-hydroxyphenyl)methylene]-1,2-diaminoethane]-manganese-(III)-chloride, [N,N′-bis[(2-hydroxy-5-sulfonatophenyl)methylene]-1,2-diaminoethane]-manganese-(III)-chloride, manganese oxalate, nitropentammine cobalt(III) chloride, nitritopentammine cobalt(III) chloride, hexammine cobalt(III) chloride, chloropentammine cobalt(III) chloride and the peroxo complex [(NH₃)₅Co—O—O—Co(NH₃)₅]Cl₄.

The agents according to the invention can contain one or more surfactants, with anionic surfactants, non-ionic surfactants and mixtures thereof being particularly suitable. Suitable non-ionic surfactants are in particular alkyl glycosides and ethoxylation and/or propoxylation products of alkyl glycosides or linear or branched alcohols each having 12 to 18 C atoms in the alkyl portion and 3 to 20, preferably 4 to 10, alkyl ether groups. Corresponding ethoxylation and/or propoxylation products of N-alkyl amines, vicinal diols, fatty acid esters and fatty acid amides which, with regard to the alkyl portion, correspond to the long-chain alcohol derivatives mentioned, and of alkyl phenols having 5 to 12 C atoms in the alkyl functional group can also be used.

Suitable anionic surfactants are in particular soaps and those containing sulfate or sulfonate groups preferably having alkali ions as cations. Usable soaps are preferably the alkali salts of saturated or unsaturated fatty acids having 12 to 18 C atoms. Fatty acids of this kind may also be used in a not completely neutralized form. Usable sulfate-type surfactants include the salts of sulfuric acid half-esters of fatty alcohols having 12 to 18 C atoms and the sulfation products of the mentioned non-ionic surfactants having a low degree of ethoxylation. Usable sulfonate-type surfactants include linear alkylbenzene sulfonates having 9 to 14 C atoms in the alkyl portion, alkane sulfonates having 12 to 18 C atoms, and olefin sulfonates having 12 to 18 C atoms, resulting from the reaction of corresponding monoolefins with sulfur trioxide, and alpha-sulfo fatty acid esters, resulting from the sulfonation of fatty acid methyl or ethyl esters.

Such surfactants are contained in the cleaning or washing agents according to the invention in proportions of preferably 5 wt. % to 50 wt. %, in particular 8 wt. % to 30 wt. %, while the agents according to the invention for cleaning hard surfaces, in particular dishes, preferably contain 0.1 wt. % to 20 wt. %, in particular 0.2 wt. % to 5 wt. %, surfactants.

An agent according to the invention preferably contains at least one water-soluble and/or water-insoluble, organic and/or inorganic builder. The water-soluble organic builder substances include polycarboxylic acids, in particular citric acid, saccharic acids and carboxymethylinuline, monomeric and polymeric aminopolycarboxylic acids, in particular glycinediacetic acid, methylglycinediacetic acid, nitrilotriacetic acid, iminodisuccinates such as ethylenediamine-N,N′-disuccinic acid and hydroxyiminodisuccinate, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds such as dextrin, and polymeric (poly)carboxylic acids, polycarboxylates which can be obtained in particular by oxidizing polysaccharides, polymeric acrylic acids, methacrylic acids, maleic acids, and mixed polymers thereof, which may also contain, polymerized in the polymer, small portions of polymerizable substances, without a carboxylic acid functionality. The relative average molecular mass (here and in the following: weight average) of the homopolymers of unsaturated carboxylic acids is generally between 5,000 g/mol and 200,000 g/mol, that of the copolymers between 2,000 g/mol and 200,000 g/mol, preferably 50,000 g/mol to 120,000 g/mol, in each case based on free acid. A particularly preferred acrylic acid—maleic acid copolymer has a relative average molecular mass of 50,000 g/mol to 100,000 g/mol. Compounds of this class which are suitable, although less preferred, are copolymers of acrylic acid or methacrylic acid with vinyl ethers, such as vinyl methyl ethers, vinyl esters, ethylene, propylene and styrene, in which the proportion of the acid is at least 50 wt. %. It is also possible to use, as water-soluble organic builder substances, terpolymers which contain two unsaturated acids and/or the salts thereof as monomers and vinyl alcohol and/or a vinyl alcohol derivative or a carbohydrate as the third monomer. The first acid monomer or the salt thereof is derived from a monoethylenically unsaturated C₃-C₈ carboxylic acid and preferably from a C₃-C₄ monocarboxylic acid, in particular from (meth)acrylic acid. The second acid monomer or the salt thereof can be a derivative of a C₄-C₈ dicarboxylic acid, maleic acid being particularly preferred. In this case, the third monomeric unit is formed of vinyl alcohol and/or preferably an esterified vinyl alcohol. In particular, vinyl alcohol derivatives are preferred which are an ester of short-chain carboxylic acids, for example C₁-C₄ carboxylic acids, with vinyl alcohol. Preferred polymers contain 60 wt. % to 95 wt. %, in particular 70 wt. % to 90 wt. %, (meth)acrylic acid or (meth)acrylate, particularly preferably acrylic acid or acrylate, and maleic acid or maleate, and 5 wt. % to 40 wt. %, preferably 10 wt. % to 30 wt. %, vinyl alcohol and/or vinyl acetate. Very particularly preferred are polymers in which the weight ratio of (meth)acrylic acid or (meth)acrylate to maleic acid or maleate is between 1:1 and 4:1, preferably between 2:1 and 3:1, and in particular between 2:1 and 2.5:1. Both the amounts and the weight ratios are based on the acids. The second acid monomer or the salt thereof can also be a derivative of an allylsulfonic acid which is substituted in the 2 position with an alkyl functional group, preferably with a C₁-C₄ alkyl functional group, or an aromatic functional group which is preferably derived from benzene or benzene derivatives. Preferred terpolymers contain 40 wt. % to 60 wt. %, in particular 45 to 55 wt. %, (meth)acrylic acid or (meth)acrylate, particularly preferably acrylic acid or acrylate, 10 wt. % to 30 wt. %, preferably 15 wt. % to 25 wt. %, methallylsulfonic acid or methallylsulfonate, and 15 wt. % to 40 wt. %, preferably 20 wt. % to 40 wt. %, of a carbohydrate as the third monomer. This carbohydrate may be, for example, a mono-, di-, oligo- or polysaccharide, mono-, di- or oligosaccharides being preferred. Sucrose is particularly preferred. The use of the third monomer presumably incorporates predetermined breaking points into the polymer which are responsible for the good biodegradability of the polymer. These terpolymers generally have a relative average molecular mass between 1,000 g/mol and 200,000 g/mol, preferably between 200 g/mol and 50,000 g/mol. Further preferred copolymers are those which have acrolein and acrylic acid/acrylic acid salts or vinyl acetate as monomers. The organic builder substances may, in particular for the preparation of liquid agents, be used in the form of aqueous solutions, preferably in the form of 30 to 50 wt. % aqueous solutions. All mentioned acids are generally used in the form of the water-soluble salts thereof, in particular alkali salts thereof.

Organic builder substances of this kind can, if desired, be contained in amounts of up to 40 wt. %, in particular up to 25 wt. %, and preferably of 1 wt. % to 8 wt. %. Amounts close to the stated upper limit are preferably used in pasty or liquid, in particular water-containing, agents.

In particular polyphosphates, preferably sodium triphosphate, are suitable as water-soluble inorganic builder materials. In particular crystalline or amorphous, water-dispersible alkali aluminosilicates are used as water-insoluble inorganic builder materials in amounts no greater than 25 wt. %, preferably of 3 wt. % to 20 wt. %, and in particular in amounts of 5 wt. % to 15 wt. %. Of these, the crystalline sodium aluminosilicates of washing agent quality, particularly zeolite A, zeolite P, and zeolite MAP, and optionally zeolite X, are preferred. Amounts close to the stated upper limit are preferably used in solid particulate agents. Suitable aluminosilicates have in particular no particles having a particle size greater than 30 μm and preferably consist of at least 80 wt. % of particles having a size smaller than 10 μm. The calcium binding capacity of said aluminosilicates is generally in the range of 100 to 200 mg CaO per gram.

In addition or as an alternative to said water-insoluble aluminosilicate and alkali carbonate, further water-soluble inorganic builder materials may be contained. These include, in addition to the polyphosphates such as sodium triphosphate, in particular the water-soluble crystalline and/or amorphous alkali silicate builders. Water-soluble inorganic builder materials of this kind are contained in agents according to the invention preferably in amounts of 1 wt. % to 20 wt. %, in particular 5 wt. % to 15 wt. %. The alkali silicates that are suitable as builder materials preferably have a molar ratio of alkali oxide to SiO₂ of less than 0.95, in particular of 1:1.1 to 1:12, and may be present in amorphous or crystalline form. Preferred alkali silicates are sodium silicates, in particular amorphous sodium silicates, having a Na₂O:SiO₂ molar ratio of 1:2 to 1:2.8. Crystalline phyllosilicates of the general formula Na₂SiₓO₂ₓ₊₁.yH₂O, in which x, referred to as the module, is a number from 1.9 to 4, y is a number from 0 to 20, and preferred values for x are 2, 3 or 4, are preferably used as crystalline silicates, which may be present alone or in a mixture with amorphous silicates. Preferred crystalline phyllosilicates are those in which x in the stated general formula assumes the values 2 or 3. Both β- and δ-sodium disilicates (Na₂Si₂O₅.yH₂O) are particularly preferred. Practically water-free crystalline alkali silicates of the above general formula, in which x is a number from 1.9 to 2.1 and which are produced from amorphous alkali silicates, may also be used in agents according to the invention. In a further preferred embodiment of agents according to the invention, a crystalline sodium phyllosilicate having a module of from 2 to 3, as can be prepared from sand and soda, is used. Sodium silicates having a module in the range of from 1.9 to 3.5 are used in a further embodiment of agents according to the invention. In a preferred embodiment of agents according to the invention, a granular compound of alkali silicate and alkali carbonate is used, as is commercially available, for example, under the name Nabion® 15.

Machine dishwashing detergents according to the invention are preferably low-alkaline and contain the usual alkali carriers such as alkali silicates, alkali carbonates and/or alkali hydrogen carbonates. The commonly used alkali carriers include carbonates, hydrogen carbonates and alkali silicates with a molar ratio SiO₂/M₂O (M=alkali atom) of 1.5:1 to 2.5:1. Alkali silicates can be present in amounts of up to 30 wt. %, based on the total agent. The use of the highly alkaline metasilicates as alkali carriers is preferably dispensed with entirely. The alkali carrier system preferably used in the agents according to the invention is a mixture of carbonate and hydrogen carbonate, preferably sodium carbonate and hydrogen carbonate, which is contained in an amount of up to 60 wt. %, preferably 10 wt. % to 40 wt. %. Depending on which pH is ultimately desired, the ratio of carbonate and hydrogen carbonate used varies, but usually an excess of sodium hydrogen carbonate is used, so that the weight ratio between hydrogen carbonate and carbonate is generally 1:1 to 15:1.

In a further embodiment of agents according to the invention for cleaning dishes, these contain 20 wt. % to 40 wt. % water-soluble organic builders, in particular alkali citrate, 5 wt. % to 15 wt. % alkali carbonate and 20 wt. % to 40 wt. % alkali disilicate.

In particular, enzymes from the class of proteases, lipases, cutinases, amylases, pullulanases, xylanases, hemicellulases, cellulases, peroxidases and oxidases or mixtures thereof are suitable as enzymes optionally contained in the agents according to the invention, the use of protease, amylase, lipase and/or cellulase being particularly preferred. The proportion is preferably 0.2 wt. % to 1.5 wt. %, in particular 0.5 wt. % to 1 wt. %. The enzymes can be adsorbed in the usual way on carrier substances and/or embedded in coating substances or incorporated as concentrated liquid formulations that are as water-free as possible.

Suitable graying inhibitors or soil release active ingredients are cellulose ethers, such as carboxymethyl cellulose, methyl cellulose, hydroxyalkyl celluloses and mixed cellulose ethers, such as methyl hydroxyethyl cellulose, methylhydroxypropyl cellulose and methyl carboxymethyl cellulose. Sodium carboxymethyl cellulose and mixtures thereof with methyl cellulose are preferably used. The soil release active ingredients commonly used include copolyesters which contain dicarboxylic acid units, alkylene glycol units and polyalkylene glycol units. The proportion of graying inhibitors and/or soil release active ingredients in agents according to the invention is generally no greater than 2 wt. % and is preferably 0.5 wt. % to 1.5 wt. %.

For example, derivatives of diaminostilbene disulfonic acid or the alkali metal salts thereof can be contained as optical brighteners in particular for textiles made of cellulose fibers (for example cotton). Salts of 4,4′-bis(2-anilino-4-morpholino-1,3,5-triazinyl-6-yl-amino)stilbene-2,2′-disulfonic acid or compounds having a similar structure which, instead of the morpholino group, have a diethanolamino group, a methylamino group or a 2-methoxyethylamino group are suitable, for example. Furthermore, brighteners of the substituted 4,4′-distyryl-diphenyl type can be present, for example 4,4′-bis-(4-chloro-3-sulfostyryl)-diphenyl. Mixtures of brighteners can also be used. Brighteners of the 1,3-diaryl-2-pyrazoline type, for example 1-(p-sulfoamoylphenyl)-3-(p-chlorophenyl)-2-pyrazoline, and compounds having a similarly structure are particularly suitable for polyamide fibers. The content of optical brighteners or brightener mixtures in the agent is generally no greater than 1 wt. %, preferably 0.05 wt. % to 0.5 wt. %. In a preferred embodiment of the invention, the agent is free from such active ingredients.

The customary foam regulators that can be used in the agents according to the invention include, for example, polysiloxane-silicic acid mixtures, the finely divided silicic acid contained therein preferably being silanized or otherwise hydrophobized. The polysiloxanes can consist of both linear compounds and crosslinked polysiloxane resins and mixtures thereof. Further defoamers are paraffinic hydrocarbons, in particular microparaffins and paraffin waxes of which the melting point is above 40° C., saturated fatty acids or soaps having in particular 20 to 22 carbon atoms, for example sodium behenate, and alkali salts of phosphoric acid mono- and/or dialkyl esters, in which the alkyl chains each have 12 to 22 carbon atoms. Among these, sodium monoalkyl phosphate and/or dialkyl phosphate having C₁₆ to C₁₈ alkyl groups is preferably used. The proportion of foam regulators can preferably be 0.2 wt. % to 2 wt. %.

The organic solvents that can be used in the agents according to the invention, in particular when the agents are present in liquid or pasty form, include alcohols having 1 to 4 C atoms, in particular methanol, ethanol, isopropanol, and tert-butanol, diols having 2 to 4 C atoms, in particular ethylene glycol and propylene glycol, and mixtures thereof, and the ethers that can be derived from the mentioned compound classes. Water-miscible solvents of this kind are present in the agents according to the invention preferably in amounts of no greater than 20 wt. %, in particular of 1 wt. % to 15 wt. %.

In order to set a desired pH that does not result automatically from mixing the other components, the agents according to the invention can contain acids that are compatible with the system and environment, in particular citric acid, acetic acid, tartaric acid, malic acid, lactic acid, glycolic acid, succinic acid, glutaric acid, and/or adipic acid, but also mineral acids, in particular sulfuric acid or alkali hydrogen sulfates, or bases, in particular ammonium or alkali hydroxides. pH regulators of this kind are contained in the agents according to the invention preferably no greater than 10 wt. %, in particular of 0.5 wt. % to 6 wt. %.

The preparation of the solid agents according to the invention presents no difficulties and can be carried out in a manner known in principle, for example by spray drying or granulation, the peroxygen compound and bleach catalyst optionally being added later separately.

Agents according to the invention in the form of solutions typically containing aqueous or other conventional solvents are particularly advantageously prepared by simple mixing of the ingredients, which can be put into an automatic mixer in bulk or as a solution.

The agents according to the invention are preferably in the form of powdery, granular or tablet-like preparations, which can be prepared in a manner known per se, for example by mixing, granulating, roller compacting and/or by spray drying the thermally loadable components and admixing the more sensitive components, which include in particular enzymes, bleaching agents and bleach-activating compounds. For the preparation of agents according to the invention having an increased bulk density, in particular in the range of 650 g/l to 950 g/l, a method having an extrusion step is preferred.

In order to prepare agents in tablet form, the preferred procedure is to mix all of the constituents with one another in a mixer and to compress the mixture by means of conventional tablet presses, for example eccentric presses or rotary presses, with applied pressures in the range of 200·10⁵ Pa to 1,500·10⁵ Pa. This readily yields break-resistant tablets that nonetheless dissolve sufficiently quickly under usage conditions, normally with a flexural strength of above 150 N. A tablet thus produced preferably has a weight of 15 g to 40 g, in particular of 20 g to 30 g, with a diameter of 35 mm to 40 mm.

The preparation of agents according to the invention in the form of non-dusting, storage-stable, free-flowing powders and/or granules with high bulk densities in the range of from 800 g/l to 1,000 g/l can also be carried out in that in a first method stage the builder components are mixed with at least one proportion of liquid mixture components to increase the bulk density of this premix and then—if desired after intermediate drying—the other constituents of the agent, including the performance-enhancing active ingredient or the performance-enhancing active ingredient combination, are combined with the premix obtained in this way.

### EXAMPLES

### Example 1

### Preparation of Bleach-Enhancing Compounds

a): Preparation of N,N,N-triethyl-N-(4-((2-oxoazepan-1-yl)carbonyl)phenyl) ethanaminium chloride (not according to the invention) (G. Wang, G. de Aragano Umbuzeiro, J. Aparecida Vendemiatti, A. Caloto de Oliveira, F. Inforcato Vacchi, M. Hussain, P. J. Hauser, H. S. Freeman, D. Hinks, J. Surfact. Deterg. 20 (2017), 277-285; S-H. Lim, N. Ç. Gursoy, P. Hauser, D. Hinks, Color. Technol. 120, (2004), 114-118)

a) i): A solution of 37.6 g of 4-chloromethyl benzoyl chloride (199 mmol) in 20 ml of toluene was added dropwise to a solution, heated to boiling under reflux in an N₂ atmosphere, of 22.5 g of caprolactam (199 mmol) and 30.4 g of triethylamine (1.5 equivalents) in 250 ml of toluene. The reaction mixture was heated to boiling under reflux with stirring for 6 hours, then allowed to cool to room temperature and filtered. The filtrate was stored in the refrigerator for 12 hours, and the precipitate which had separated out was filtered off, washed with 5% strength aqueous NaHCO₃ solution and dried at 40° C. for 12 hours. 47.6 g (179 mmol; 90%) of 1-((4-(chloromethyl)phenyl)carbonyl)azepan-2-one were obtained as a colorless solid.
¹H-NMR (400 MHz, DMSO-d₆): δ=1.85 (bs, 6H), 2.67-2.73 (m, 2H), 3.94-4.01 (m, 2H), 4.59 (s, 2H), 7.41 (pd, J=8.5 Hz, 2H), 7.53 (pd, J=8.5 Hz, 2H) ppm.

a) ii): A solution of 24.1 g of triethylamine (240 mmol) in 10 ml of acetonitrile was added dropwise in an N₂ atmosphere to a solution of 60 g of ((4-(chloromethyl)phenyl)carbonyl)azepan-2-one (230 mmol), prepared according to a) i), in 350 ml of acetonitrile and the reaction mixture was heated to boiling under reflux with stirring for 4 hours. It was then cooled to room temperature and the solvent was removed on a rotary evaporator. 40 ml of acetone were added to the residue, the mixture was heated to 50-60° C. for 1 hour, then the acetone was removed by filtration and the residue was dried. 58.9 g (161 mmol; 72%) of N,N,N-triethyl-N-(4-((2-oxoazepan-1-yl)carbonyl)phenyl)ethanaminium chloride were obtained as a colorless powder.
¹H-NMR (400 MHz, CDCl₃): δ=1.37 (t, J=7.2 Hz, 9H), 1.74-1.85 (bs, 6H), 2.60-2.68 (m, 2H), 3.35 (q, J=7.2 Hz, 6H), 3.92 (bs, 2H), 4.82 (s, 2H), 7.47 (pd, J=8, 4 Hz, 2H), 7.63 (pd, J=8.4 Hz, 2H) ppm.

### b) Preparation of 1-methyl-3-(4-((2-oxopyrrolidin-1-yl)carbonyl)benzyl)-1H-imidazol-3-ium chloride

b) i): A solution of 75.6 g of 4-chloromethyl benzoyl chloride (400 mmol) in 50 ml of toluene was added dropwise to a solution, heated to boiling under reflux in an N₂ atmosphere, of 34.0 g of 2-oxopyrrolidin (400 mmol) and 60.7 g of triethylamine (1.5 equivalents) in 250 ml of toluene. The reaction mixture was heated to boiling under reflux with stirring for 6 hours, then allowed to cool to room temperature and filtered. The filtrate was stored in the refrigerator for 12 hours, and the precipitate which had separated out was filtered off, washed with 5% strength aqueous NaHCO₃ solution and dried at 40° C. for 12 hours. 13.8 g (58 mmol; 29%) of 1-((4-(chloromethyl)phenyl)carbonyl)pyrrolidin-2-one were obtained as a colorless solid.
¹H-NMR (400 MHz, DMSO-d₆): δ=1.75-1.95 (m, 4H, CH₂), 3.60-3.80 (m, 2H, CH₂), 4, 81 (s, 2H, CH₂), 7.41-7.49 (m, 2H, Ar—H), 7.51-7.59 (m, 2H, Ar—H) ppm.

b) ii) A solution of 4.76 g of 1-methylimidazole (57.8 mmol) in 15 ml of acetonitrile was added dropwise in an N₂ atmosphere to a solution of 13.1 g of 1-((4-(chloromethyl)phenyl)carbonyl)pyrrolidin-2-one (55 mmol), prepared according to b) i), in 60 ml of acetonitrile and the reaction mixture was heated to boiling under reflux with stirring for 4 hours. It was then cooled to room temperature and the solvent was removed on a rotary evaporator. 20 ml of acetone were added to the residue, the mixture was heated to 50-60° C. for 1 hour, then the acetone was removed by filtration and the process was repeated three times. Finally, the residue was dried at 40° C. overnight. 16.8 g (52.2 mmol; 95%) of 1-methyl-3-(4-((2-oxopyrrolidin-1-yl)carbonyl)benzyl)-1H-imidazol-3-ium chloride were obtained as colorless solid.
FTIR (film): [symbol] =3090, 3007, 1740, 1704, 1661, 1562, 1421, 1359, 1303, 1230, 1188, 1174, 1161, 1021, 738, 625, 534 cm⁻¹.
¹H-NMR (400 MHz, DMSO-d₆): δ=2.04 (tt, J=7.7 Hz, 7.6 Hz, 2H), 2.53 (t, J=7.9 Hz, 2H), 3.81 (t, J=7.1 Hz, 2H), 3.87 (s, 3H), 5.51 (s, 2H), 7.44 (pd, J=8.3 Hz, 2H), 7.59 (pd, J=8.3 Hz, 2H), 7.76 (t, J=1.8 Hz, 1H), 7.85 (t, J=1.8 Hz, 1H), 9.34 (s, 1H) ppm.
¹³C-NMR (100.6 MHz, DMSO-d₆): δ=17.0 (t), 32.6 (t), 35.8 (q), 46.0 (t), 51.1 (t), 122.3 (d), 123.9 (d), 127.4 (d, 2C), 129.0 (d, 2C), 135.0 (s), 136.9 (s), 138.0 (d), 169.2 (s), 174.7 (s).
MS (ESI positive mode, +3.5 kV): 284 ([M−Cl⁺]⁺, 100%).

### c) Preparation of 1-methyl-3-(4-((2-oxo-1,3-thiazolidin-3-yl)carbonyl)benzyl)-1H-imidazol-3-ium chloride

c) i): A solution of 75.6 g of 4-chloromethyl benzoyl chloride (400 mmol) in 50 ml of toluene was added dropwise to a solution, heated to boiling under reflux in an N₂ atmosphere, of 41.3 g of 1,3,thiazolidin-2-one (200 mmol) and 60.7 g of triethylamine (1.5 equivalents) in 450 ml of toluene. The reaction mixture was heated to boiling under reflux with stirring for 6 hours, then allowed to cool to room temperature and filtered. The filtrate was stored in the refrigerator for 12 hours, and the precipitate which had separated out was filtered off, washed with 5% strength aqueous NaHCO₃ solution and dried at 40° C. for 12 hours. 37.6 g (147 mmol; 51%) of 3-((4-(chloromethyl)phenyl)carbonyl)-1,3-thiazolidin-2-one were obtained as a colorless solid.
FTIR (film): [symbol] =2949, 2892, 1703, 1670, 1611, 1357, 1290, 1224, 1153, 1018, 965, 848, 800, 765, 722, 671, 651, 479 cm⁻¹.
¹H-NMR (400 MHz, CDCl₃): δ=3.39 (t, J=7.0 Hz, 2H), 4.23 (t, J=7.0 Hz, 2H), 4.60 (s, 2H), 7.43 (pd, J=8.1 Hz, 2H), 7.62 (pd, J=8.5 Hz, 2H) ppm.
¹³C-NMR (CDCl₃, 100.6 MHz): δ=25.8 (t), 45.4 (t), 48.3 (t), 128.0 (d, 2C), 129.4 (d, 2C), 133.5 (s), 141.5 (s), 169.1 (s), 172.3 (s).

c) ii) A solution of 23.8 g of 1-methylimidazole (290 mmol) in 20 ml of acetonitrile was added dropwise in an N₂ atmosphere to a solution of 37.2 g of 3-((4-(chloromethyl)phenyl)carbonyl)-1,3-thiazolidin-2-one (145 mmol), prepared according to c) i), in 200 ml of acetonitrile and the reaction mixture was heated to boiling under reflux with stirring for 4 hours. It was then cooled to room temperature and the solvent was removed on a rotary evaporator. 40 ml of acetone were added to the residue, the mixture was heated to 50-60° C. for 1 hour, then the acetone was removed by filtration and the process was repeated three times. Finally, the residue was dried at 40° C. overnight. 37.2 g (110 mmol; 76%) of 1-methyl-3-(4-((2-oxo-1,3-thiazolidin-3-yl)carbonyl)benzyl)-1H-imidazol-3-ium chloride were obtained as a colorless solid.
FTIR (film): [symbol] >=3149, 3006, 2944, 2852, 1779, 1697, 1668, 1306, 1284, 1228, 1156, 964, 844, 807, 735, 684, 624, 613, 470 cm⁻¹.
¹H-NMR (400 MHz, D₂O): δ=3.52 (t, J=7.1 Hz, 2H), 3.97 (s, 3H), 4.27 (t, J=7, 1 Hz, 2H), 5.51 (s, 2H), 7.52 (pd, J=8.4 Hz, 2H), 7.55 (s, 2H), 7.70 (pd, J=8, 4 Hz, 2H), 8.90 (s, 1H) ppm.
¹³C-NMR (D₂O, 100.6 MHz): δ=28.6 (t), 38.6 (q), 51.5 (t), 54.9 (t), 125.1 (d), 126.7 (d), 131.0 (d, 2C), 132.1 (d, 2C), 136.9 (s), 139.8 (s), 140.7 (s), 173, 2 (s), 179.1 (s) ppm.
MS (ESI positive mode, +3.5 kV): 302 ([M−Cl⁻]⁺, 100%).

### Example 2

### Decolorizing Test

98 mg of sodium percarbonate and 13.1 mg of TAED or one of the compounds prepared in Examples 1 a), 1 b) and 1 c) were added to a solution, adjusted to pH 10-11 by the addition of NaOH, of 2.5 mg morin in 100 ml of water and stirred at room temperature. The time taken for the yellow color to completely disappear was determined and is shown in Table 1.

TABLE 1
Time until complete decolorizing

| Active | Duration |
|---|---|
| ingredient | [minute] |
| TAED | 210 |
| Compound from | 130 |
| Example 1 b) | |
| Compound from | 110 |
| Example 1 c) | |
| Compound from | 270 |
| Example 1 a) | |

### Example 3

### Washing Attempts

Washing tests were carried out at 30° C. with standardized stains on cotton specified in Table 3, using a washing liquor with 3.8 g/l each of a solid washing agent of the composition specified in Table 2 below, agents M1 and M2 being the compounds according to the invention prepared in Examples 1 b) and c); for comparison, agent V2 contained the non-inventive compound from Example 1 a), and agent V1 did not contain such a compound, but rather the conventional bleach activator TAED. The evaluation was carried out by measuring the color distance according to the L*a*b* values and the Y values calculated therefrom as a measure of the brightness. Table 3 shows the differences in the Y values that resulted after washing between V1 and M1, V1 and M2, and V1 and V2.

TABLE 2
Composition (wt. %)

| | V1 | M1 | M2 | V2 |
|---|---|---|---|---|
| Na alkylbenzene sulfonate | 13 | 13 | 13 | 13 |
| Na fatty alcohol sulfate | 3 | 3 | 3 | 3 |
| Na carboxymethylcellulose | 3 | 3 | 3 | 3 |
| HEDP-Na4 | 1 | 1 | 1 | 1 |
| Na polyacrylate | 3 | 3 | 3 | 3 |
| Sodium silicate | 7 | 7 | 7 | 7 |
| Sodium carbonate | 21 | 21 | 21 | 21 |
| Sodium hydrogen carbonate | 10 | 10 | 10 | 10 |
| Sodium percarbonate | 12 | 12 | 12 | 12 |
| TAED | 3.1 | — | — | — |
| Compound from Example 1 b) | — | 3.1 | — | — |
| Compound from Example 1 c) | — | — | 3.1 | — |
| Compound from Example 1 a) | — | — | — | 3.1 |
| Water, sodium sulfate, perfume, dye | | | | |
| to make up to 100 | | | | |

TABLE 3
ΔY values

| Stain/agent | M1 | M2 | V2 |
|---|---|---|---|
| Red wine | 5.9 | 5.8 | 4.5 |
| Coffee | 4.0 | 2.4 | ND |
| Beetroot | 2.8 | 3.0 | ND |
| Apple | 2.3 | 1.7 | 1.0 |
| Blackcurrant | 1.2 | 3.4 | ND |
| Cherry juice | 2.6 | 2.7 | 1.9 |

ND: not determined
The brightness values when the active ingredients essential to the invention are used are greater than those which result when the washing agents containing other bleach activators are used.

### Example 4

### Cleaning Tests

0.5 g of a mixture of lactose and ovalbumin (weight ratio 3:1) was stirred in 10 ml of water at room temperature for 10 minutes, then the mixture was freeze-dried, ground in a mortar and heated to 160° C. for 20 minutes. Mixtures of 15 mg in each case of the burnt-on food residue-simulating dirt obtained in this way, 3.75 mg of sodium percarbonate and 0.5 mg of a compound prepared in Example 1 b) or 1 c) in 5 ml of water were heated to 60° C. for 48 hours in a mechanical shaker. After the durations given in Table 4, the dissolution of the artificial dirt was visually evaluated and rated on a scale from 0 (no dissolution) to ++++ (completely dissolved).

TABLE 4
Dissolving burnt-on dirt

| Active ingredient/ | | | | | | | |
|---|---|---|---|---|---|---|---|
| duration | 0.5 h | 1 h | 2 h | 4 h | 6 h | 24 h | 48 h |
| None | 0 | 0 | 0 | 0 | 0 | 0 | 0 |
| Compound from | + | + | ++ | +++ | +++ | +++ | ++++ |
| Example 1 b) | | | | | | | |
| Compound from | 0 | 0 | + | + | + | + | ++ |
| Example 1 c) | | | | | | | |

What is claimed is:

1. A bleach-activating compound of general formula (I), 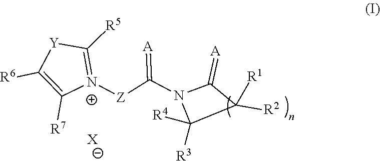
in which
A represents O, S, Se;
Z represents an optionally substituted C₁₋₃₀ alkylene, C₃₋₃₀ cycloalkylene, arylene, alkylenearylene or arylenealkylene functional group;
X⁻ represents an anion;
Y represents NR⁸, O, S, Se;
R¹, R², R³, R⁴, R⁵, R⁶, R⁷ and R⁸ represent, independently of one another, H, a C₁₋₃₀ alkyl, C₃₋₃₀ cycloalkyl, aryl, alkylaryl or arylalkyl functional group; and
n represents a number in the range from 0 to 12,
wherein 1 or more non-adjacent carbon atoms not bonded to a heteroatom can be replaced by a heteroatom in the hydrocarbon functional groups.

2. The bleach-activating compound of claim 1, wherein the anion X⁻ is selected from the group consisting of chloride, bromide, iodide, tosylate, mesylate, triflate, sulfate, carbonate, and phosphate.

3. The bleach-activating compound of claim 1, wherein in the hydrocarbon functional groups 1 or more non-adjacent carbon atoms not bonded to a heteroatom can be replaced by N, O, S and/or Se.

4. The bleach-activating compound of claim 1, wherein the compound of general formula (I) corresponds to general formula (II), 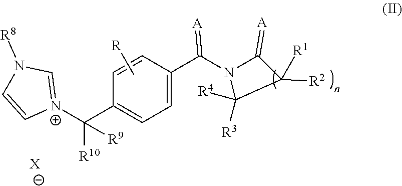
in which R, R⁹ and R¹⁰ represent, independently of one another, H, a C₁₋₃₀ alkyl, C₃₋₃₀ cycloalkyl, aryl, alkylaryl or arylalkyl functional group, wherein 1 or more non-adjacent carbon atoms not bonded to a heteroatom can be replaced by a heteroatom.

5. A method for washing laundry or a method for cleaning hard surfaces, comprising the method steps of
(a) providing an aqueous liquor containing H₂O₂ or an inorganic peroxygen compound which provides H₂O₂ in water and a compound according to general formula (I), 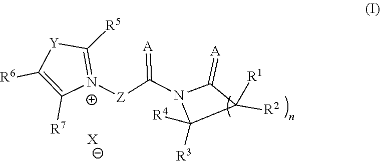
in which
A represents O, S, Se;
Z represents an optionally substituted C₁₋₃₀ alkylene, C₃₋₃₀ cycloalkylene, arylene, alkylenearylene or arylenealkylene functional group;
X⁻ represents an anion;
Y represents NR⁸, O, S, Se;
R¹, R², R³, R⁴, R⁵, R⁶, R⁷ and R⁸ represent, independently of one another, H, a C₁₋₃₀ alkyl, C₃₋₃₀ cycloalkyl, aryl, alkylaryl or arylalkyl functional group; and
n represents a number in the range from 0 to 12;
wherein 1 or more non-adjacent carbon atoms not bonded to a heteroatom can be replaced by a heteroatom in the hydrocarbon functional groups, and
(b) bringing this liquor into contact with a textile to be washed or a hard surface to be cleaned.

6. The method according to claim 5, wherein the anion X⁻ is selected from the group consisting of chloride, bromide, iodide, tosylate, mesylate, triflate, sulfate, carbonate, and phosphate.

7. The method according to claim 5, wherein in the hydrocarbon functional groups 1 or more non-adjacent carbon atoms not bonded to a heteroatom can be replaced by N, O, S and/or Se.

8. The method according to claim 5, wherein the contact between the aqueous liquor and the textile to be washed or the hard surface to be cleaned is made at temperatures in the range of 20° C. to 40° C.

9. The method according to claim 5, wherein the contact between the aqueous liquor and the textile to be washed or the hard surface to be cleaned is made at temperatures in the range of 20° C. to 30° C.

10. The method according to claim 5, wherein the textile to be washed or the hard surface to be cleaned remains in contact with the aqueous liquor over a period of 20 minutes to 120 minutes.

11. The method according to claim 5, wherein the textile to be washed or the hard surface to be cleaned remains in contact with the aqueous liquor over a period of 30 minutes to 90 minutes.

12. The method according to claim 5, wherein the compound of general formula (I) corresponds to general formula (II), 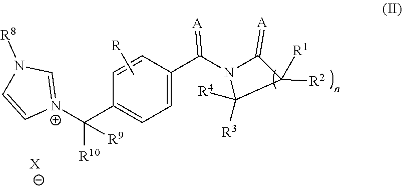
in which R, R⁹ and R¹⁰ represent, independently of one another, H, a C₁₋₃₀ alkyl, C₃₋₃₀ cycloalkyl, aryl, alkylaryl or arylalkyl functional group, wherein 1 or more non-adjacent carbon atoms not bonded to a heteroatom can be replaced by a heteroatom.

13. The method according to claim 5, wherein the amounts of H₂O₂ or peroxygen compounds are selected such that the liquor includes 10 ppm to 10% active oxygen.

14. The method according to claim 5, wherein the amount of H₂O₂ or peroxygen compounds are selected such that the liquor includes 50 ppm to 5,000 ppm active oxygen.

15. A washing or cleaning agent containing a compound according to general formula (I), 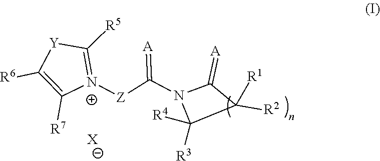
in which
A represents O, S, Se;
Z represents an optionally substituted C₁₋₃₀ alkylene, C₃₋₃₀ cycloalkylene, arylene, alkylenearylene or arylenealkylene functional group;
X⁻ represents an anion;
Y represents NR⁸, O, S, Se;
R¹, R², R³, R⁴, R⁵, R⁶, R⁷ and R⁸ represent, independently of one another, H, a C₁₋₃₀ alkyl, C₃₋₃₀ cycloalkyl, aryl, alkylaryl or arylalkyl functional group; and
n represents a number in the range from 0 to 12;
wherein 1 or more non-adjacent carbon atoms not bonded to a heteroatom can be replaced by a heteroatom in the hydrocarbon functional groups.

16. The washing agent or cleaning agent according to claim 15, wherein the anion X⁻ is selected from the group consisting of chloride, bromide, iodide, tosylate, mesylate, triflate, sulfate, carbonate, and phosphate.

17. The washing agent or cleaning agent according to claim 15, wherein in the hydrocarbon functional groups 1 or more non-adjacent carbon atoms not bonded to a heteroatom can be replaced by N, O, S and/or Se.

18. The washing agent or cleaning agent according to claim 15, wherein the agent includes 0.01 wt. % to 50 wt. %, of the compound according to general formula (I).

19. The washing agent or cleaning agent according to claim 15, wherein the agent includes 0.1 wt. % to 25 wt. % of the compound according to general formula (I).

20. The washing agent or cleaning agent according to claim 15, wherein the compound of general formula (I) corresponds to general formula (II), 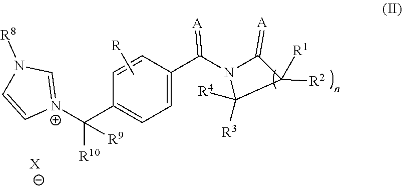
in which R, R⁹ and R¹⁰ represent, independently of one another, H, a C₁₋₃₀ alkyl, C₃₋₃₀ cycloalkyl, aryl, alkylaryl or arylalkyl functional group, wherein 1 or more non-adjacent carbon atoms not bonded to a heteroatom can be replaced by a heteroatom, in these functional groups.
